(19) United States
(12) Patent Application Publication
(10) Pub. No.: US 2025/0297986 A1
(43) Pub. Date: Sep. 25, 2025

(54) GAS ANALYZER

(71) Applicant: Baker Hughes Holdings LLC, Houston, TX (US)

(72) Inventors: Aniruddha Weling, Wayland, MA (US); Michael Collins, Broadford (IE); Yufeng Huang, Andover, MA (US); Anthony Kowal, Berlin, MA (US)

(21) Appl. No.: 19/080,683

(22) Filed: Mar. 14, 2025

Related U.S. Application Data
(60) Provisional application No. 63/567,210, filed on Mar. 19, 2024.

(51) Int. Cl. G01N 27/74; G01N 33/00

(52) U.S. Cl. CPC G01N 27/74; G01N 33/0006

(57) ABSTRACT

A method for determining gas concentrations using a gas sensor configured to analyze a gas sample containing oxygen and a binary background gas. The method includes determining calibration ranges for each gas component of a binary background gas, receiving first and second voltage outputs corresponding to the oxygen and the background gas, and verifying that the voltage outputs fall within the calibration range. A first ratio is computed based on differences between the first voltage output and calibration voltages for the oxygen in the first and second gas components. Similarly, a second ratio is computed based on the second voltage output. The concentration of oxygen in the gas sample is determined by solving an equation equating the first and second ratios. The concentrations of the first and second gas components are then determined from the oxygen concentration and the computed ratios. The determined gas concentrations are subsequently provided as output.

### CROSS-REFERENCE TO RELATED APPLICATIONS

This application claims priority to U.S. Provisional Patent Application No. 63/567,210 filed on Mar. 19, 2024, and entitled “GAS ANALYZER,” the disclosure of which is incorporated herein by reference in its entirety.

### BACKGROUND

Gas analyzers can be deployed in various industrial settings to determine types and concentrations of gases present within those settings. Sensors within the gas analyzers can include electrical circuits configured to generate and convey signal data associated with one or more gases being sensed by the sensor. Accurately and efficiently determining the type and concentration of gases present in a gaseous mixture in real time can be important for generating reliable process data and for monitoring hazardous conditions in industrial settings.

### SUMMARY

In an aspect, a method is provided. In an embodiment, the method can include determining a calibration range for each gas of a plurality of gases analyzed via a gas sensor. The gas sensor is configured to receive a gas sample comprising oxygen and a binary background gas. The binary background gas includes a first gas component and a second gas component. The method also includes receiving data characterizing a first voltage output associated with a first portion of a circuit of the gas sensor and a second voltage output associated with a second portion of the circuit of the gas sensor. The first voltage output and the second voltage output corresponds to the oxygen and the binary background gas in the gas sample respectively. The method also includes determining that the first and second voltage outputs are within the respective calibration ranges by comparing the first and second voltage outputs to calibration voltages corresponding to an unknown amount of the oxygen in the first gas component and the second gas component respectively. The method also includes computing, based on the determining, a first ratio characterizing a difference between the first voltage output and a first calibration voltage corresponding to the unknown amount of the oxygen in the first gas component and a difference between the calibration voltages corresponding to the unknown amount of the oxygen in the first and second gas components respectively. The first ratio corresponds to a ratio of a concentration of the first gas component to a concentration of the second gas component in the gas sample, based on the first voltage output. The method also includes computing, based on the determining, a second ratio characterizing a difference between the second voltage output and a second calibration voltage corresponding to the unknown amount of the oxygen in the first gas component and a difference between the calibration voltages corresponding to the unknown amount of the oxygen in the first and second gas components respectively. The second ratio corresponds to a ratio of the concentration of the first gas component to the concentration of the second gas component in the gas sample, based on the second voltage output. The method also includes determining a concentration of the oxygen in the gas sample by solving the equation obtained by equating the first ratio and the second ratio. The method also includes determining the concentration of the first and the second gas component in the gas sample from the concentration of the oxygen, the first ratio, and the second ratio. The method further includes providing the concentration of the oxygen, the first gas component, and the second gas component based on the determining.

The disclosed method can be implemented in a variety of ways. For example, in one aspect, it can be implemented within a system that includes at least one data processor and a non-transitory memory storing instructions for the processor to perform aspects of the method. Alternatively, or in addition, the method can be included in non-transitory computer readable memory storing the method as instructions which, when executed by at least one data processor forming part of at least one computing system, causes the at least one data processor to perform operations of the method.

In another embodiment, the first gas component has a first thermal conductivity that differs from a second thermal conductivity of the second gas component. In another embodiment, the binary background gas can include two of nitrogen, carbon dioxide, methane, hydrogen, helium, or argon. In another embodiment, the binary gas mixture can include a biogas.

In another embodiment, determining the calibration range for each gas can include generating calibration tables having calibration ranges corresponding to a known amount of oxygen in each gas component of the binary background gas. Each one of the calibration tables includes the calibration voltages. Each one of the calibration tables maps the first calibration voltage associated with the first portion of the circuit of the gas sensor to a corresponding oxygen concentration when the oxygen is present in the first gas component or the second gas component, and the second calibration voltage associated with the second portion of the circuit of the gas sensor to a corresponding oxygen concentration when the oxygen is present in the first gas component or the second gas component.

In another embodiment, the gas sensor can be a thermoparamagnetic sensor.

In another embodiment, a first sensor response time constant corresponding to the first gas component and a second sensor response time constant corresponding to the second gas component can be identified from analyzing an uncorrected response of the gas sensor to a step change in the concentration of the oxygen in the first and second gas components. A combined sensor response time constant associated with the binary background gas can be determined as a weighted function of the first sensor response time constant and the second sensor response time constant based on the concentration of the first and second gas components in the gas sample.

In another embodiment, the concentration of the oxygen in the gas sample containing two background gases can be determined, based on the combined sensor response time constant, in a reduced sensor response time (e.g., less than 90 seconds).

### DESCRIPTION OF DRAWINGS

These and other features will be more readily understood from the following detailed description taken in conjunction with the accompanying drawings, in which:

FIG. 1 illustrates an exemplary embodiment of a sensing system configured for sensing a gas sample emitted from a source.

FIG. 2 illustrates an exemplary embodiment of a thermoparamagnetic sensor of the sensing system of FIG. 1.

FIG. 3 illustrates an arrangement of sensing elements configured within a magnetic field generated by the thermoparamagnetic gas sensor in FIG. 2.

FIG. 4 illustrates a signal generation circuit configured within the gas sensor in FIG. 2.

FIG. 5 illustrates an exemplary embodiment of a method for determining a concentration and/or a type of gas included in the gas sample using the sensing system of FIG. 1.

FIG. 6 illustrates a plurality of calibration tables used for determining the concentration and/or the type of gas included in the gas sample using the sensing system of FIG. 1 according to the method of FIG. 5.

FIG. 7 illustrates a plurality of calibration tables and equations used for determining the concentration and/or the type of gas included in the gas sample using the sensing system of FIG. 1 according to the method of FIG. 5.

FIG. 8 shows a plurality of equations used for determining the concentration and/or the type of background gas included in the gas sample using the sensing system of FIG. 1 according to the method of FIG. 5.

FIG. 9 illustrates how the sensing system of FIG. 1 responds to a step change in oxygen concentration in the gas sample.

### DETAILED DESCRIPTION

Gas analyzers can be configured in various industrial settings to analyze a gas sample and to determine the type and concentration of gases present in the gas sample. Typically, to determine the concentration or type of gas present in a gas sample, users need to acquire separate analyzers for sensing different gas types that may be present within the gas sample. For example, one analyzer can be configured to measure oxygen within the gas sample. A second analyzer can be configured to measure a background gas present in the gas sample. The cost and logistical burden of acquiring, configuring, and operating different gas analyzers to determine the concentration and/or type of gases in gas samples can be significant for maintaining multiple types of gas analyzers in a desired sampling environment.

An improved method of determining the concentration and/or type of gases in a gas sample is provided. The method can be performed using a single gas analyzer to reduce the overall logistical burden of performing different gas measurements of the gas sample. The improved method can provide a novel way of using calibration data associated with the gas analyzer to distinguish oxygen from the gas sample and to determine a concentration and/or type of one or more background gases that may be included in the gas sample. The gas analyzer can be configured to determining the concentration and/or type of the background gas based on an inverse relationship between temperature and magnetic susceptibility of oxygen. As a result of this inverse relationship, heating a portion of an oxygen-containing mixture in a non-homogenous magnetic field can create a “magnetic wind’ within the gas sensor. The magnetic wind, along with gas thermal conductivity, can be measured via its thermal effect on thermistors configured within the gas sensor. The change in electrical resistance of thermistors arranged in a Wheatstone bridge circuit can provide data signals corresponding to measurements of the gas sample, such as oxygen concentration and a background gas concentration in the gas sample as the gas sample flows through the gas sensor. These measurements can be used to generate calibration tables, which can be further used to estimate both oxygen concentration and background gas concentration in real-time as the gas analyzer receives the gas sample.

Advantageously, measuring oxygen and background gas concentrations simultaneously provides the opportunity for users to reduce their cost and operating expense in measuring concentrations of different gases using different types of gas analyzers. Additionally, the methods described herein can utilize the calibration data to determine gas types and concentrations thereof faster and in near real-time as compared to existing legacy systems requiring multiple gas analyzers that can be configured to identify and measure concentrations of single gases.

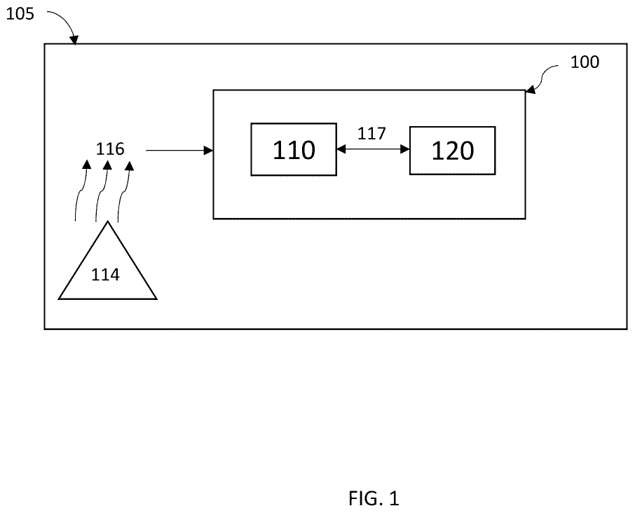

FIG. 1 is a diagram illustrating a sensing environment 105 in which a gas emission source 114 can be present and emitting a gas sample 116. The gas sample 116 can include a mixture of gases that can emanate from the emission source 114. The emission source 114 can be located within various industrial environments from which the gas 116 can be emitted or otherwise released. In some embodiments, the industrial environments can include an oil or gas refinery, a chemical manufacturing environment, or a piece of industrial equipment, such as a compressor or a turbine. In some embodiments, the industrial environment can include a biogas production facility, a biogas generator, an animal breeding facility, such as poultry, swine, or livestock farm. The gas sample 116 can include a single gas or a gaseous mixture. In some embodiments, the gas sample 116 can include oxygen, nitrogen, carbon dioxide, methane, or the like. In some embodiments, the gas sample 116 can include a binary background gas that includes a mixture of two gases with oxygen, such as a gas that includes a mixture of oxygen, carbon dioxide, and methane.

As shown in FIG. 1 a sensing system 100 can be configured within environment 105. In some aspects, the sensing system 100 can include a gas sensor 110 and a gas analyzer 120 communicably coupled to the gas sensor 110. In some embodiments, the gas sensor 110 can include a thermoparamagnetic gas sensor. The gas analyzer 120 can be configured as a computing device including at least one data processor and at least one memory configured to receive data from the gas sensor 110 and to determine a concentration and/or a type of background gas included in the gas sample 116. In some embodiments, the gas sensor 110 and the gas analyzer 120 can be collocated within a housing of the gas sensor 110.

As illustrated in FIG. 1, the gas sample 116 can be received or otherwise sampled by the gas sensor 110. The gas sensor 110 can include a plurality of signal generating elements in an electrical circuit that can generate a measurement signal describing the concentration and/or type of gas present in the sample gas mixture 116. The signal generating elements can include a configuration of circuits that can include a constant temperature electrical measurement bridge, such as a Wheatstone bridge of thermistors. The measurement bridge of the gas sensor 110 can include a series of wind sensing and wind generating thermistors that, when heated or cooled, can generate a signal proportional to the temperature difference between them. The gas sensor can also include a magnetic field with a gradient that peaks in the center of the sensor cell. The gas sample 116 can flow through this magnetic field with its gas properties which are either diamagnetic or paramagnetic. Gases that are diamagnetic are repelled by a magnetic field. Gases that are paramagnetic are attracted to a magnetic field. Since oxygen is present in the sample gas mixture 116, its high paramagnetic susceptibility allows it to be attracted to the magnetic field.

The magnetic field can be generated in a continuous manner and can be applied to the received gas sample 116. Wind sensing and wind generating thermistors can be heated to elevated temperatures by an electric current from the constant temperature electrical bridge and can create a temperature gradient. The oxygen content in the gas sample can cause a magnetic wind to flow from the wind generating thermistors to the wind sensing thermistors, and hence, it can be evaluated based on the paramagnetic or diamagnetic properties of different gases present in the gas sample 116.

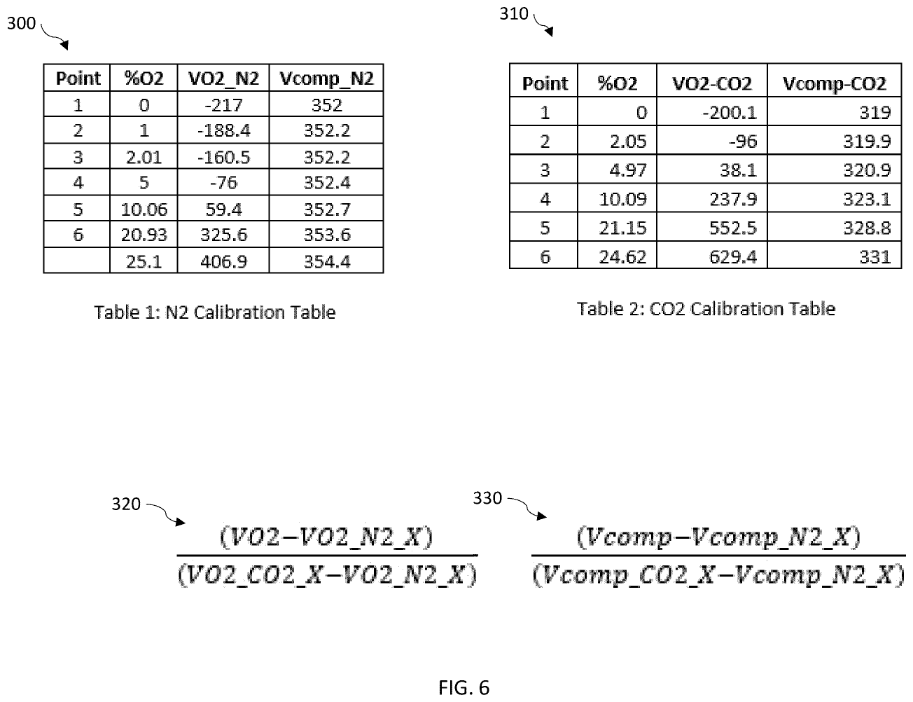

As the gas sample 116 flows through the sensor 110, the temperature of the wind sensing and wind generating thermistors can change. For example, if oxygen is present in the gas sample 116 a high pressure can be created near the wind generating thermistors thereby causing the magnetic wind to cool the wind generating thermistors, which experience a lower temperature as they lose heat. Therefore, the wind sensing thermistors can be warmed up slightly due to heat passed by the magnetic wind from the wind generating thermistors. The temperature difference between the wind generating thermistors and wind sensing thermistors can be picked up as a signal proportional to the oxygen content in the gas sample. Furthermore, heat loss from the thermistors to the gas sample 116 varies with the gas sample's thermal conductivity, and to maintain the thermistors at constant temperature, an outer constant temperature electrical bridge needs to adjust the amount of power supplied to the thermistors so as to cause the thermistors to remain at a constant temperature. The signal proportional to the oxygen content from the inner bridge and the signal driving power adjustments associated with maintaining the temperature of the thermistors constant by the constant temperature outer electrical bridge can correspond to voltage outputs characterizing the presence of oxygen and/or background gas present in the gas sample 116. The voltage outputs can be communicated to the gas analyzer 120 by means of signals 117 from the sensor 110 and can be stored in a memory of the gas analyzer 120 as a calibration table as shown in FIG. 6.

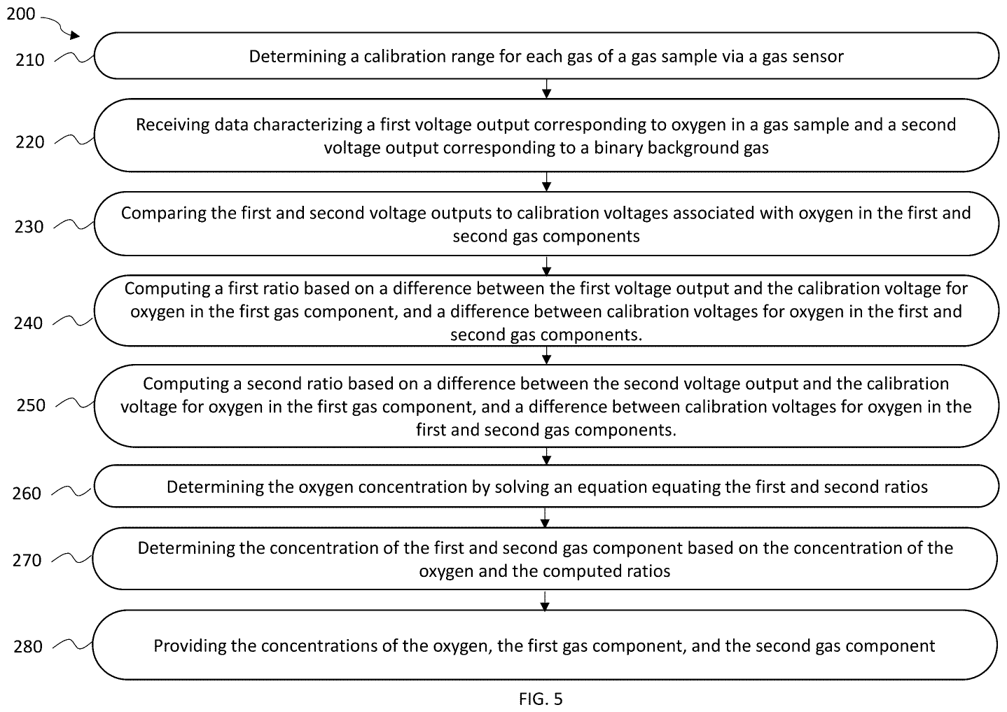

The gas analyzer 120 can include multiple inter-connected components, such as a data processor, a memory, a controller, and a display. In some embodiments, the display can include a graphical user interface (GUI). The data processor can be configured to execute computer-readable and executable instructions stored in the memory to perform the method 200 described in relation to FIG. 5. The memory can further store data associated with the signal output 117, as well as sensor setpoint values associated with operation of the gas sensor 110. The gas analyzer 120 can also execute computer-readable instructions stored in the memory, which cause the data processor to control operation of the gas sensor 110 via control signals 117. In this way, the controller can control operation of the gas sensor 110 based on measurement data values generated by the gas sensor 110 as determined by the data processor.

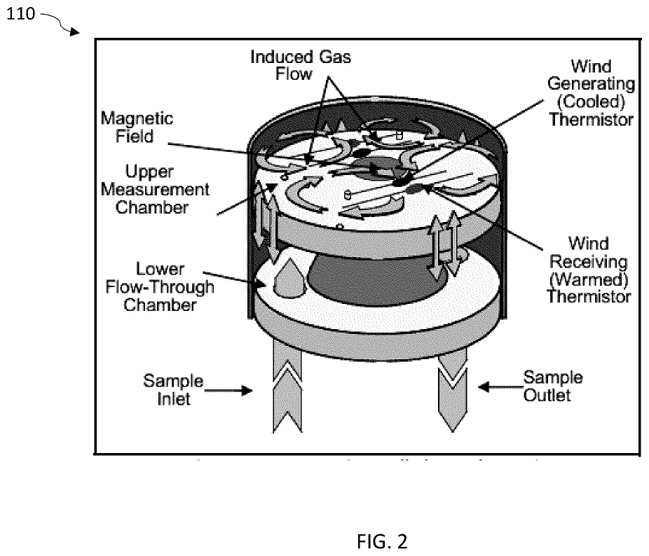

FIG. 2 illustrates an example gas sensor 110 of the sensing system 100 shown in FIG. 1. The gas sensor 110 can generate a magnetic field through which the gas sample 116 can flow through as described above. The gas sensor 110 can be configured to receive the gas sample 116 emitted from the source 114 through the sample inlet as shown in FIG. 2. The gas sensor 110 can include permanent magnets that create a magnetic field. The entire sensor cell including the upper and lower measurement chambers can be heated to maintain its temperature constant at about 45° C. (113° F.) to achieve thermal equilibrium independent of the ambient temperature. As described above, the sensor 110 can contain a series of thermistors that are either wind generating or wind sensing. In some embodiments, the thermistors can be glass-coated to enhance their lifetime. In some embodiments, the thermistors can be arranged in matched pairs. One pair can reside inside the magnetic field while another pair can reside outside the magnetic field. The thermistors can be electrically heated to a constant temperature and can thus create a temperature gradient only when there is gas flow within the magnetic field.

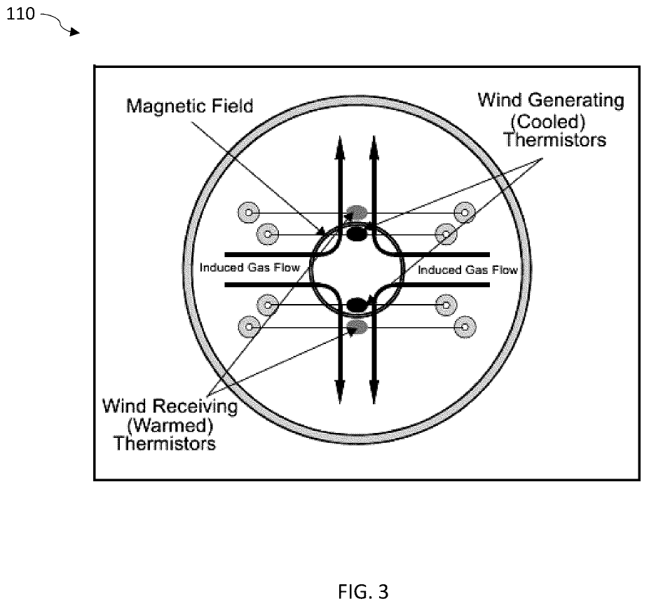

FIG. 3 is a top-down view of a gas sensor 110 illustrating the arrangement of thermistor pairs in the gas sensor 110. If the gas sample 116 contains a paramagnetic gas such as oxygen, it is attracted to the magnetic field, causing the sample gas pressure to become locally higher in the center of the chamber. At the same time, the sample gas pressure is slightly lower near the thermistors because the high thermistor temperature causes the paramagnetic properties of oxygen to decrease. This slight gradient in sample gas pressure causes the sample gas to flow outward from the center of the magnetic field and over the thermistors. As a result, the inner, wind-generating thermistors decrease in temperature as they lose heat to the magnetic wind. This causes a temperature gradient between the cooler inner thermistors and the warmer outer thermistors, which in turn leads to an imbalance between their resistance values as they are initially matched when no gas is flowing.

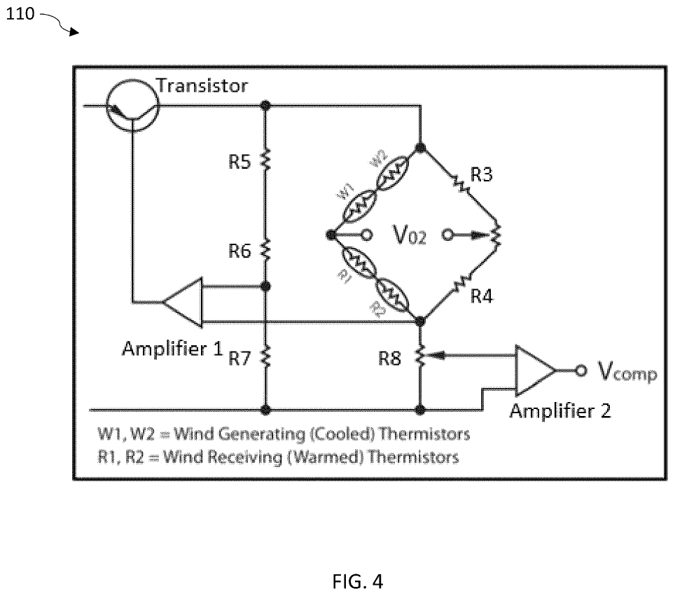

FIG. 4 illustrates how the two thermistor pairs can be connected in series in an inner Wheatstone bridge circuit. When the gas sample 116 flows through the sensor with an oxygen containing mixture, the bridge circuit can become unbalanced as the electrical resistance of the thermistors changes with temperature. This circuit imbalance can create a voltage drop, which is proportional to the oxygen concentration in the gas being measured, to appear across the bridge circuit. The diamond shaped inner bridge, also known as a Wheatstone bridge, can be used for measurement of the oxygen and can be considered a first portion of a read-out circuit. W1 and W2 refer to the heat generating thermistors and R1 and R2 refer to the heat sensing thermistors. Each pair R1, R2, and W1, W2 can be configured to form two sides of the Wheatstone bridge. If the gas sample 116 contains no oxygen, the Wheatstone bridge is balanced, thereby making (R1+R2)/(W1+W2)=R4/R3, and VO2=0. When oxygen is present, the magnetic wind changes the ratio of (R1+R2)/(W1+W2), by reducing the temperature of (W1+W2) and increasing the temperature of (R1+R2). In this case, the Wheatstone bridge is no longer balanced, and VO2 is used as the voltage output proportional to the oxygen concentration for oxygen measurement.

Also shown in FIG. 4 is the outer, rectangle-shaped Wheatstone bridge which can be used for background gas measurement and can be considered a second portion of the read-out circuit. The four sides of this outer rectangle bridge can comprise of resistors (R5+R6), R7, the inner bridge as described above and R8. The outer rectangle bridge is always kept balanced by a feedback loop consisting of the Amplifier 1 and Transistor. As a gas sample 116, comprising of an oxygen containing mixture, flows through the gas sensor 110, the background gas can be measured by its thermal conductivity. For instance, if a background gas has a high thermal conductivity, the transistor will need to provide a higher current through the thermistor elements, W1, W2, R1 and R2 in order to maintain them at a constant temperature. The voltage required to maintain this constant temperature can be referred to as Vcomp and can be used as the output for background gas concentration measurement. The signal output described herein can be communicated from gas sensor 110 to gas analyzer 120 by means of signal output 117. Additionally, the signal output Vcomp can be stored in a calibration table as shown in FIG. 6.

FIG. 5 is a diagram illustrating a method 200 for determining a concentration and/or type of the background gas present in the gas sample 116 according to the subject matter described herein. In some embodiments, the method 200 can include a step 210 of determining a calibration range for each gas of a plurality of gases via a gas sensor. The plurality of voltage outputs can correspond to a first voltage output from the inner diamond bridge portion of the gas sensor related to the concentration of oxygen in a first background gas. The calibration range can further include a plurality of voltage outputs related to the concentration of oxygen in a second gas of a binary background gas. The plurality of voltages can correspond to a second voltage output from the outer rectangle-shaped Wheatstone bridge portion of the circuit. In some respects, the step 210 involves generating a calibration table for each gas being analyzed. For example, the calibration table can include a calibrated range of voltage outputs corresponding to the concentration of oxygen in one or more background gases.

The method 200 can also include a step 220 of receiving data characterizing a first voltage output corresponding to oxygen in a gas sample and a second voltage output corresponding to a background gas in the gas sample. The first voltage output can occur due to the change in temperature of a pair of thermistors due to the oxygen content in the gas sample associated with an unbalanced bridge circuit. The second voltage output can occur due to a thermal conductivity associated with the background gas, thereby causing a transistor of a circuit to provide sufficient electric current in order to maintain the temperature of a pair of thermistors in the inner bridge. The circuit described herein can include a Wheatstone bridge circuit.

The method 200 can also include a step 230 of determining that the first and second voltage outputs are within the respective calibration ranges of oxygen in each background gas component by comparing the first and second voltage outputs to calibration voltages corresponding to an unknown amount of the oxygen in the first gas component and the second gas component respectively. To enable real-time compensation of change in sensor response due to changes in the binary background gas composition, the system can be calibrated over the desired range of oxygen in both gas components of the binary background gas.

For example, FIG. 6 illustrates sample calibration tables 300 and 310. Tables 300 and 310 include voltage outputs corresponding to the presence of oxygen in a first gas component such as nitrogen and oxygen in a second gas component such as carbon dioxide, e.g., table 300 corresponding to “Nitrogen Calibration Table 1” and table 310 corresponding to “CO2 Calibration Table 2”. The gas sensor 110 can generate two independent voltages for a given gas sample—a voltage output of the inner bridge corresponding to VO2, and Vcomp, a voltage output from the outer bridge corresponding to a thermal conductivity of a background gas.

The two gas components in the background gas should have a significant difference in their thermal conductivity at the operating temperature of the gas sensor. The calibration tables as shown in FIG. 6 includes calibration ranges corresponding to a known amount of oxygen (% O₂) in each gas component. Since the response of the gas sensor is non-linear, values of the known amount of oxygen at which each sensor is calibrated in either gas component can be chosen to obtain best accuracy and linearity over a given calibration range. Each calibration table as shown in FIG. 6 includes a first calibration voltage (VO2) associated with the first portion (inner bridge) of the read-out circuit mapped to a corresponding oxygen concentration when the oxygen is present in the first gas component or the second gas component, and a second calibration voltage (Vcomp) associated with the second portion (outer bridge) of the read-out circuit mapped to a corresponding oxygen concentration when the oxygen is present in the first gas component or the second gas component. During the calibration procedure, values of both the first and the second calibration voltages are recorded as a function of the oxygen level (% O₂).

The method 200 can also include a step 240 of computing, based on the determining, a first ratio characterizing a difference between the first voltage output and the calibration voltage corresponding to the unknown amount of the oxygen (X) in the first gas component and a difference between the first calibration voltages corresponding to the unknown amount of the oxygen (X) in the first and second gas components respectively. The first ratio corresponds to a ratio of a concentration of the first gas component to a concentration of the second gas component in the gas sample, based on the first voltage output. In other words, the first ratio, as shown in expression 320, represents a relationship between the concentration of the first gas component e.g., nitrogen and the concentration of the second gas component e.g., carbon dioxide in the gas sample 116 being analyzed in terms of the first voltage output (VO2).

The method 200 can also include a step 250 of computing, based on the determining, a second ratio characterizing a difference between the second voltage output and the calibration voltage corresponding to the unknown amount of the oxygen (X) in the first gas component and a difference between the second calibration voltages corresponding to the unknown amount of the oxygen (X) in the first and second gas components respectively. The second ratio corresponds to a ratio of the concentration of the first component to the concentration of the second component in the gas sample, based on the second voltage output. In other words, the second ratio, as shown in expression 330, represents a relationship between the concentration of the first gas component e.g., nitrogen and the concentration of the second gas component e.g., carbon dioxide in the gas sample 116 being analyzed in terms of the second voltage output (Vcomp). In some aspects of step 240 and step 250, the first ratio and/or the second ratio can be determined using linear interpolation based on respective calibration tables.

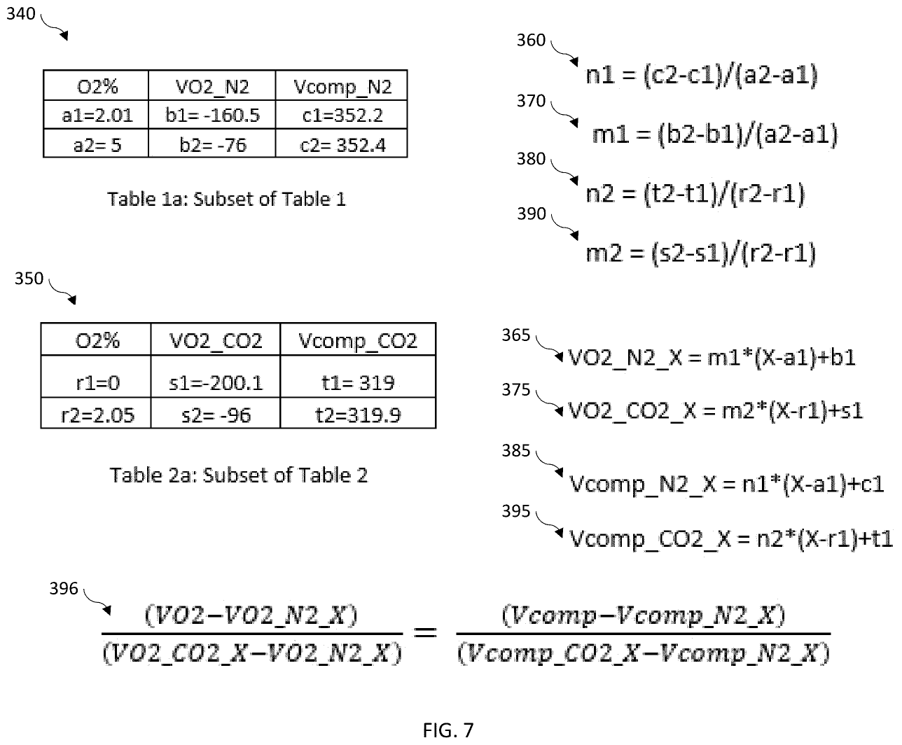

The method 200 can also include a step 260 of determining a concentration of the oxygen in the gas sample. For example, as described above in relation to FIG. 4, the gas analyzer 120 can utilize both the first and second ratios determined via expressions 320 and 330, as shown in FIG. 6, to determine if the VO2 and Vcomp outputs are in the range of the calibrated voltages defined in the calibration tables 300, 310. The equation 396 as shown in FIG. 7 equating 320 and 330 can be used to determine both the oxygen concentration and the background gas concentration in the gas sample. These expressions can be utilized due to the linear relationship between the oxygen concentration X and VO2 signal corresponding to oxygen concentration as well as the Vcomp signal corresponding to the thermal conductivity of the background gas for non-interacting gases in the gas sample. The concentration of the oxygen (X) is then determined by solving the equation 396.

In detail, as shown in FIG. 7, the equation 396 describes the process of comparing voltage outputs VO2 and Vcomp in a gas sample wherein the gas sample can be calibrated in a nitrogen-carbon dioxide gas mixture. The equation 396 can utilize the tables 340 and 350 which provide a subset of calibration values corresponding to the calibration tables 300 and 310, respectively. The subset of calibration values can enable the determination a first ratio of the first voltage output and the first calibration voltage through using the equations 370 and 390. For instance, the voltage output VO2_N2, also referred to as “b1” and “b2” in equation 370, can represent the calibrated voltage outputs measuring oxygen in a gas mixture where oxygen and nitrogen are present, and wherein the calibrated voltage output VO2_N2 can be closely related to the real-time voltage output VO2 in value. VO2_CO2, also referred to as “s1” and “s2” in equation 390, can represent the calibrated voltage output measuring oxygen in a gas mixture where oxygen and carbon dioxide are present, and wherein the calibrated voltage output VO2_CO2 can be closely related to the real-time voltage output VO2 in value. The values of a1 and a2, in equation 370, can represent the calibrated concentration of oxygen in a gas sample comprising oxygen and nitrogen. The values of r1 and r2, in equation 390, can represent the calibrated concentration of oxygen in a gas sample comprising oxygen and carbon dioxide.

The ratio of equation 370, also referred to as “m1”, can represent the first step in determining a first ratio of the first voltage output, and first calibration voltage of a gas including oxygen and a background gas comprising nitrogen. The ratio of equation 390, also referred to here as “m2”, can represent the second step in determining a first ratio of the first voltage output and a first calibration voltage of a gas including oxygen and a background gas comprising carbon dioxide. Based on the ratio m1, the gas analyzer 120 can calculate the voltage output VO2 as a function of the unknown oxygen concentration “X”, and use equation 365 along with the ratio m1 to yield a value VO2_N2_X. Based on the ratio m2, the gas analyzer 120 can calculate the voltage output VO2, as a function of the unknown oxygen concentration “X”, and use equation 375 along with ratio m2 to yield a value VO2_CO2_X. Both VO2_N2_X and VO2_CO2_X can be inserted into the ratio in equation 320 and can represent the determining a first voltage output such as oxygen is in a calibrated range of a first gas including oxygen and a binary background gas comprising nitrogen and carbon dioxide.

The equations 360 and 380 can utilize the tables 340 and 350 which provide a subset of calibration values corresponding to the calibration tables 300 and 310, respectively, to determine if Vcomp is in the range of the calibration voltages. The subset of calibration values can enhance the determination of a second ratio of the second voltage output and the second calibration voltage. For instance, the voltage output Vcomp can represent the real-time voltage output measuring one gas component of a binary background gas. Vcomp_N2, also referred to as “c1” and “c2”, can represent the calibrated output of the outer bridge in a binary background gas where oxygen and nitrogen are present, and wherein the calibrated voltage output Vcomp_N2 can be closely related to the real-time voltage output Vcomp in value. Vcomp_CO2, also referred to as “t1” and “t2”, can represent the calibrated voltage of the outer bridge in a gas mixture where oxygen and carbon dioxide are present, and wherein the calibrated voltage output VO2_CO2 can be closely related to the real-time voltage output VO2 in value.

The ratio of equation 360, also referred to as “n1”, can represent the first step in determining a second ratio of the second voltage output, and second calibration voltage of a gas including oxygen and a binary background gas containing nitrogen. The ratio of equation 380, also referred to here as “n2”, can represent the second step in determining a second ratio of the second voltage output and a second calibration voltage of a gas including oxygen and a binary background gas containing carbon dioxide. Based on the ratio n1, the gas analyzer 120 can calculate the voltage output Vcomp, and use equation 385 along with the ratio n1 to yield a value Vcomp_N2_X. Based on the ratio n2, the gas analyzer 120 can calculate the voltage output Vcomp, and use equation 395 along with ratio n2 to yield a value Vcomp_CO2_X. Both Vcomp values, Vcomp_N2_X and Vcomp_CO2_X can be inserted into the ratio in expression 330 and can represent the determining a second voltage output is in a calibrated range of the background gas containing oxygen, nitrogen, and carbon dioxide.

Furthermore, the first ratio as shown in expression 320 and the second ratio as shown in expression 330 can be inserted into equation 396 which can provide a value representing the concentration of oxygen X in the gas sample 116. The measured value VO2 can represent an unknown concentration of oxygen, also referred to here as “X”, and the gas analyzer 120 can solve for X using the known values calculated as described above.

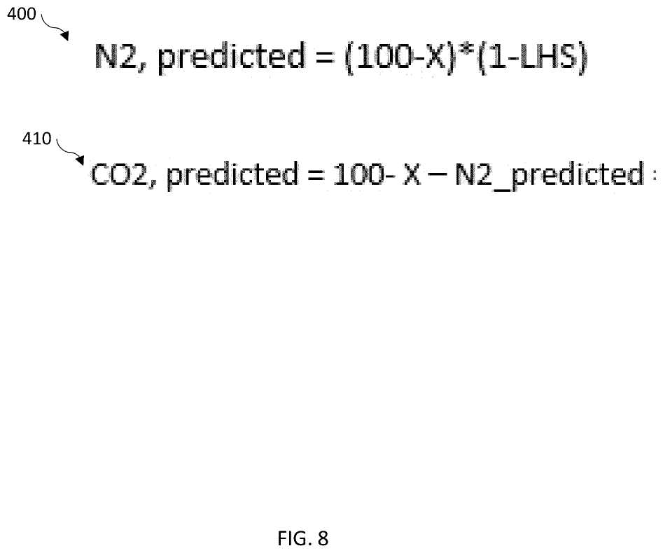

The method 200 can also includes a step 270 of determining the concentration of the first and second gas component in the gas sample from the concentration of the oxygen, the first ratio, and the second ratio determined in previous steps 260, 240, and 250. For example, as shown in FIG. 8, once the gas analyzer 120 predicts the value X, it can then insert this value into equation 400 along with the first ratio (expression 320), also referred to here as the left hand side “LHS”, therefore providing a value of the concentration of a first gas component, such as nitrogen, in the binary background gas. Once the gas analyzer 120 receives this value from equation 400, the gas analyzer 120 can insert the concentration of the first gas in equation 410, therefore providing a value of the concentration of a second gas component, such as carbon dioxide, in the binary background gas. At step 280, the concentration of the oxygen, the first gas component, and the second gas component can then be provided, for example, to a display or a downstream processing unit for further analysis.

It should be noted that the proposed calibration method as described herein covers oxygen measurement in binary non-interacting background gas applications such as oxygen in methane/carbon dioxide mixes for biogas monitoring or oxygen in nitrogen/carbon dioxide or nitrogen/methane mixes for hydrocarbon inerting applications.

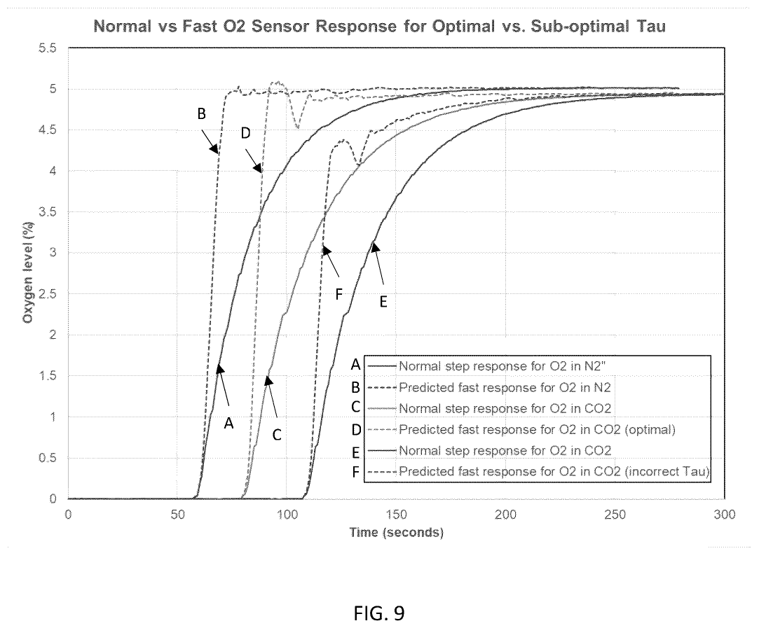

FIG. 9 illustrates how the sensing system 100, specifically the gas analyzer 120, responds to a step change in oxygen concentration in the gas sample 116 having the background gas nitrogen. As shown in FIG. 9, the solid line A represents an uncorrected raw sensor response to a change from 100% nitrogen to 5% oxygen in nitrogen. The uncorrected raw sensor response follows an exponential rise characterized by a sensor response time constant (t). The concentration of the oxygen in the gas sample 116 can be modeled as:

$${O}_{2} ( t ) = {O}_{{2}_{final}} \times ( 1 - {e}^{- \frac{t}{τ}} )$$

Where O₂_{_{final}} is the steady-state oxygen concentration, and τ represents the sensor response time constant associated with the background gas. It should be noted that the value of τ is inversely related to the thermal conductivity of the background gas especially if the oxygen level detected is relatively small.

Accordingly, the method and the system described herein improves the sensor response time by predicting the concentration measurement of oxygen using predefined or predetermined sensor response time constants (τ) for the background gas. The steady-state oxygen concentration can be predicted significantly faster, for example, as shown in FIG. 9 by the dashed line B, in under 15 seconds than the time taken for the raw sensor response to reach the steady state (e.g., over 90 seconds) using the method and the system described herein.

The variance in sensor response time constant is illustrated in FIG. 9 by the solid line C which represents an uncorrected sensor response to the same 5% oxygen step in carbon dioxide. The gas sensor takes over 120 seconds to reach a steady state. The dashed line D is the corresponding predicted sensor response in carbon dioxide calculated from this raw response using a different (higher) value of τ.

As described above, the one or more first sensor response time constant (τₓ) corresponding to a first gas component, such as nitrogen in the background gas, and one or more second sensor response time constant (τ_{y}) corresponding to a second gas component, such as carbon dioxide in the background gas can be identified from analyzing the uncorrected response of the gas sensor to a step change in the concentration of the oxygen in the first and second gas components. In some implementations, the system can determine a combined sensor response time constant based on the pre-identified first and second sensor response time constants. For example, the combined sensor response time constant is computed as a weighted function of τₓ and τ_{y} based on the relative concentrations of the first and second gas components in the binary background gas. The system can use the combined sensor response time constant to calculate the steady-state oxygen concentration of the gas sample 116 within an optimally shortened response time (e.g., less than 90 seconds). For example, the thermal conductivity of nitrogen is approximately 50% higher than that of carbon dioxide at 25° C. ambient temperature. In such binary mix of nitrogen and carbon dioxide, increasing the carbon dioxide content will effectively increase the τ values of the mix of nitrogen and carbon dioxide. Therefore, for a 50-50 mix of nitrogen and carbon dioxide, the combined sensor response time constant is an average of the τ values of nitrogen and carbon dioxide.

It should be noted that using an incorrect sensor response time constant for predicting sensor response results in unstable readings, causing the predicted concentration of the oxygen to either overshoot or undershoot the true steady-state value. For example, as illustrated in FIG. 9, the solid line E represents an uncorrected sensor response in carbon dioxide, which takes over 120 seconds to stabilize. The dashed line F shows the incorrect fast response when using τ for nitrogen instead of carbon dioxide, causing undershoot and requiring more than 60 seconds to reach the steady state. This is significantly slower than the time taken to reach the same steady state value using the correct time constant for carbon dioxide.

As indicated above, the background analysis scheme as described herein provides a method of classifying and characterizing binary background gas mixes using the dissimilar variation of Vcomp with % O2 in each component due to its distinct thermal conductivity. Therefore, if the gas analyzer 120 is calibrated in binary background gas such as nitrogen and carbon dioxide, only the nitrogen and carbon dioxide levels in the background gas are measured. Such background gas analysis typically done by an independent and more complex gas analyzer such as a gas chromatograph, but by providing the concentration of the oxygen, the first gas component, and the second gas component simultaneously, the background gas analysis performed by gas analyzer 120 can reduce the overall system cost and response time appreciably.

Exemplary technical effects of the methods, systems, and computer-readable medium described herein include, by way of non-limiting example, determining a concentration of oxygen and determining a concentration and/or type of a background gas present in a gas sample using a single gas sensor. The concentration and/or type of the background gas can be determined using one or more calibration tables determined based on a concentration of oxygen present within the gas sample. Advantageously, the methods, systems, and computer-readable mediums herein can enable determination of a broad range of gas types and concentrations using a single sensor, thereby alleviating the need for multiple disparate sensor systems.

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment can be combined with the features of other embodiments. Such modifications and variations are intended to be included within the scope of the present invention. Further, in the present disclosure, like-named components of the embodiments generally have similar features, and thus, within a particular embodiment each feature of each like-named component is not necessarily fully elaborated upon.

The subject matter described herein can be implemented in analog electronic circuitry, digital electronic circuitry, and/or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, can be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language can correspond to the precision of an instrument for measuring the value. Here and throughout the specification and claims, range limitations can be combined and/or interchanged, such ranges are identified and include all the sub-ranges contained therein unless context or language indicates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

1. A method comprising:
determining a calibration range for each gas of a plurality of gases analyzed via a gas sensor configured to receive a gas sample comprising oxygen and a binary background gas including a first gas component and a second gas component;
receiving data characterizing a first voltage output associated with a first portion of a circuit of the gas sensor and a second voltage output associated with a second portion of the circuit of the gas sensor, the first voltage output and the second voltage output corresponding to the oxygen and the binary background gas in the gas sample respectively;
determining that the first and second voltage outputs are within the respective calibration ranges by comparing the first and second voltage outputs to calibration voltages corresponding to an unknown amount of the oxygen in the first gas component and the second gas component respectively;
computing, based on the determining, a first ratio characterizing a difference between the first voltage output and a first calibration voltage corresponding to the unknown amount of the oxygen in the first gas component and a difference between the calibration voltages corresponding to the unknown amount of the oxygen in the first and second gas components respectively, wherein the first ratio corresponds to a ratio of a concentration of the first gas component to a concentration of the second gas component in the gas sample, based on the first voltage output;
computing, based on the determining, a second ratio characterizing a difference between the second voltage output and a second calibration voltage corresponding to the unknown amount of the oxygen in the first gas component and a difference between the calibration voltages corresponding to the unknown amount of the oxygen in the first and second gas components respectively, wherein the second ratio corresponds to a ratio of the concentration of the first gas component to the concentration of the second gas component in the gas sample, based on the second voltage output;
determining a concentration of the oxygen in the gas sample by solving an equation equating the first ratio and the second ratio;
determining the concentration of the first and the second gas component in the gas sample from the concentration of the oxygen, the first ratio, and the second ratio; and
providing the concentration of the oxygen, the first gas component, and the second gas component based on the determining.

2. The method of claim 1, wherein the first gas component has a first thermal conductivity that differs from a second thermal conductivity of the second gas component.

3. The method of claim 2, wherein determining the calibration range for each gas comprises:
generating calibration tables including calibration ranges corresponding to a known amount of oxygen in each gas component of the binary background gas, wherein each one of the calibration table includes the calibration voltages and maps:
the first calibration voltage associated with the first portion of the circuit of the gas sensor to a corresponding oxygen concentration when the oxygen is present in the first gas component or the second gas component; and
the second calibration voltage associated with the second portion of the circuit of the gas sensor to a corresponding oxygen concentration when the oxygen is present in the first gas component or the second gas component.

4. The method of claim 1, wherein the binary background gas includes two of nitrogen, carbon dioxide, methane, hydrogen, helium, or argon.

5. The method of claim 1, wherein the binary background gas comprises a biogas.

6. The method of claim 1, wherein the gas sensor is a thermoparamagnetic sensor.

7. The method of claim 1, further comprising:
identifying a first sensor response time constant corresponding to the first gas component and a second sensor response time constant corresponding to the second gas component from analyzing an uncorrected response of the gas sensor to a step change in the concentration of the oxygen in the first and second gas components.

8. The method of claim 7, further comprising:
determining a combined sensor response time constant associated with the binary background gas, wherein the combined sensor response time constant is determined as a weighted function of the first sensor response time constant and the second sensor response time constant based on the concentration of the first and second gas components in the gas sample.

9. The method of claim 8, wherein determining the concentration of the oxygen comprises:
determining, based on the combined sensor response time constant, the concentration of the oxygen in the gas sample within a raw sensor response time of less than 90 seconds.

10. A system comprising:
a gas sensor configured to receive a gas sample comprising oxygen and a binary background gas including a first gas component and a second gas component;
at least one data processor communicably coupled to the gas sensor; and
a memory storing instructions, which when executed by at the least one data processor causes the at least one data processor to perform operations comprising:
determining a calibration range for each gas of the gas sample;
receiving data characterizing a first voltage output associated with a first portion of a circuit of the gas sensor and a second voltage output associated with a second portion of the circuit of the gas sensor, the first voltage output and the second voltage output corresponding to the oxygen and the binary background gas in the gas sample respectively;
determining that the first and second voltage outputs are within the respective calibration ranges by comparing the first and second voltage outputs to calibration voltages corresponding to an unknown amount of the oxygen in the first gas component and the second gas component respectively;
computing, based on the determining, a first ratio characterizing a difference between the first voltage output and a first calibration voltage corresponding to the unknown amount of the oxygen in the first gas component and a difference between the calibration voltages corresponding to the unknown amount of the oxygen in the first and second gas components respectively, wherein the first ratio corresponds to a ratio of a concentration of the first gas component to a concentration of the second gas component in the gas sample, based on the first voltage output;
computing, based on the determining, a second ratio characterizing a difference between the second voltage output and a second calibration voltage corresponding to the unknown amount of the oxygen in the first gas component and a difference between the calibration voltages corresponding to the unknown amount of the oxygen in the first and second gas components respectively, wherein the second ratio corresponds to a ratio of the concentration of the first gas component to the concentration of the second gas component in the gas sample, based on the second voltage output;
determining a concentration of the oxygen in the gas sample by solving an equation equating the first ratio and the second ratio;
determining the concentration of the first and the second gas component in the gas sample from the concentration of the oxygen, the first ratio, and the second ratio; and
providing the concentration of the oxygen, the first gas component, and the second gas component based on the determining.

11. The system of claim 10, wherein the first gas component has a first thermal conductivity that differs from a second thermal conductivity of the second gas component.

12. The system of claim 11, wherein determining the calibration range for each gas comprises:
generating calibration tables including calibration ranges corresponding to a known amount of oxygen in each gas component of the binary background gas, wherein each one of the calibration table includes the calibration voltages and maps:
the first calibration voltage associated with the first portion of the circuit of the gas sensor to a corresponding oxygen concentration when the oxygen is present in the first gas component or the second gas component; and
the second calibration voltage associated with the second portion of the circuit of the gas sensor to a corresponding oxygen concentration when the oxygen is present in the first gas component or the second gas component.

13. The system of claim 10, wherein the binary background gas includes two of nitrogen, carbon dioxide, methane, hydrogen, helium, or argon.

14. The system of claim 10, wherein the binary background gas comprises a biogas.

15. The system of claim 10, wherein the gas sensor is a thermoparamagnetic sensor.

16. The system of claim 10, wherein the operations performed by the at least one data processor further comprises:
identifying a first sensor response time constant corresponding to the first gas component and a second sensor response time constant corresponding to the second gas component from analyzing an uncorrected response of the gas sensor to a step change in the concentration of the oxygen in the first and second gas components.

17. The system of claim 16, wherein the operations performed by the at least one data processor further comprises:
determining a combined sensor response time constant associated with the binary background gas, wherein the combined sensor response time constant is determined as a weighted function of the first sensor response time constant and the second sensor response time constant based on the concentration of the first and second gas components in the gas sample.

18. The system of claim 17, wherein determining the concentration of the oxygen comprises:
determining, based on the combined sensor response time constant, the concentration of the oxygen in the gas sample within a raw sensor response time of less than 90 seconds.

19. A non-transitory computer-readable medium storing instructions, which when executed by at least one data processor cause the at least one data processor to perform operations comprising:
determining a calibration range for each gas of a plurality of gases analyzed via a gas sensor configured to receive a gas sample comprising oxygen and a binary background gas including a first gas component and a second gas component;
receiving data characterizing a first voltage output associated with a first portion of a circuit of the gas sensor and a second voltage output associated with a second portion of the circuit of the gas sensor, the first voltage output and the second voltage output corresponding to the oxygen and the binary background gas in the gas sample respectively;
determining that the first and second voltage outputs are within the respective calibration ranges by comparing the first and second voltage outputs to calibration voltages corresponding to an unknown amount of the oxygen in the first gas component and the second gas component respectively;
computing, based on the determining, a first ratio characterizing a difference between the first voltage output and a first calibration voltage corresponding to the unknown amount of the oxygen in the first gas component and a difference between the calibration voltages corresponding to the unknown amount of the oxygen in the first and second gas components respectively, wherein the first ratio corresponds to a ratio of a concentration of the first gas component to a concentration of the second gas component in the gas sample, based on the first voltage output;
computing, based on the determining, a second ratio characterizing a difference between the second voltage output and a second calibration voltage corresponding to the unknown amount of the oxygen in the first gas component and a difference between the calibration voltages corresponding to the unknown amount of the oxygen in the first and second gas components respectively, wherein the second ratio corresponds to a ratio of the concentration of the first component to the concentration of the second component in the gas sample, based on the second voltage output;
determining a concentration of the oxygen in the gas sample by solving an equation equating the first ratio and the second ratio;
determining the concentration of the first and the second gas component concentrations in the gas sample from the concentration of the oxygen, the first ratio, and the second ratio; and
providing the concentration of the oxygen, the first gas component, and the second gas component based on the determining.
